(19) United States
(12) Patent Application Publication
(10) Pub. No.: US 2023/0248939 A1
(43) Pub. Date: Aug. 10, 2023

(54) MODULAR, PERCUTANEOUS DUAL-LUMEN CANNULA SYSTEM

(71) Applicants: UNIVERSITY OF MARYLAND, BALTIMORE, Baltimore, MD (US); University of Maryland Medical System Corporation, Linthicum, MD (US); The Feinstein Institutes for Medical Research, Manhasset, NY (US)

(72) Inventors: Bartley P. Griffith, Gibson Island, MD (US); Zhongjun Wu, Marriottsville, MD (US); Chetan Pasrija, Gaithersburg, MD (US); Aakash Yogesh Shah, Baltimore, MD (US); Zachary Kon, Thornwood, NY (US)

(21) Appl. No.: 18/167,475

(22) Filed: Feb. 10, 2023

Related U.S. Application Data
(60) Provisional application No. 63/308,705, filed on Feb. 10, 2022.
(60) Provisional application No. 63/308,684, filed on Feb. 10, 2022.
(60) Provisional application No. 63/330,459, filed on Apr. 13, 2022.

(51) Int. Cl. A61M 25/00; A61M 1/00; A61M 39/02

(52) U.S. Cl. CPC A61M 25/0026; A61M 1/84; A61M 39/0247

(57) ABSTRACT

A modular cannula for drainage and return of fluids from and to a body, such as the body of a patient, is adjustable to enable an operator to achieve desired flow characteristics during such fluid drainage and return procedures. The modular, dual-lumen cannula system has an outer drainage cannula and an inner return cannula that is moveable with respect to the outer drainage cannula. By enabling movement of the inner return cannula with respect to the outer drainage cannula, a distance between return outlet ports at the distal end of the return cannula and drainage ports at the distal end of the drainage cannula may be manually adjusted. Such adjustable distance allows for optimal positioning of both the drainage and return ports that are particularly adapted to an individual patient’s physiology, in turn enabling optimal fluid flow characteristics to be achieved regardless of that patient physiology.

### CROSS-REFERENCE TO RELATED APPLICATIONS

This application claims the benefit of U.S. Provisional Pat. App. No. 63/308,705 titled “Percutaneous Drainage Cannula,” filed by the inventors herein on Feb. 10, 2022, and of U.S. Provisional Pat. App. No. 63/308,684 titled “Curved Guidewire,” filed by the inventors herein on Feb. 10, 2022, and of U.S. Provisional Pat. App. No. 63/330,459 titled “Percutaneous Drainage Cannula,” filed by the inventors herein on Apr. 13, 2022, the specifications of which are incorporated herein by reference in their entireties.

### FIELD OF THE INVENTION

The present invention relates generally to cannulas for drainage and return of fluids from and to a body, and more particularly to a modular cannula system for drainage and return of fluids from and to a body, such as the body of a patient, that is adjustable to enable an operator to achieve desired flow characteristics during such fluid drainage and return procedures.

### BACKGROUND

Cannulas are often used for patient life support. Often times such cannulas are inserted into a patient’s body at differing points of entry and independently serve to drain fluid, such as blood, from a portion of the patient’s body, and to return fluid, such as blood, to the patient’s body. Such differing points of entry add to the risks associated with such procedures in addition to patient discomfort following the procedure and during recuperation. As a result, efforts have been made to employ dual-lumen cannulas in order to provide a single assembly, and thus a single point of entry into the patient’s body, that functions simultaneously as both a drainage cannula and a return cannula. Unfortunately however, current dual-lumen cannula, such as those that are designed to be percutaneously placed with the distal tip in the pulmonary artery, are bonded together resulting in a fixed distance between the drainage port or ports and the return port or ports. As patients’ height, and consequently intravascular distances, vary, the fixed distance between the drainage and the return ports often results in suboptimal positioning of those ports (and most typically the drainage ports). Suboptimal positioning typically hinders drainage and return due to flow characteristics within the vessel and cannula, and the difficulty of precisely locating the cannula, and more particularly the tip of the cannula. The resulting poor drainage and return of blood may even further risk patient health by increasing morbidity and even the risk of death.

Currently, the only product on the market known to the inventors herein that allows for a percutaneously placed coaxially aligned dual-lumen cannula is the PROTEK DUO system produced by LivaNova. This is a dual lumen cannula with an outer drainage lumen bonded to an inner return lumen intended to be positioned in a patient’s pulmonary artery. This system can be used with a ventricular assist device for right ventricular support or with a pump-oxygenator for respiratory support. However, because the system comprises bonded drainage and return lumens, the foregoing challenges concerning suboptimal placement are present, such that there remains a need in the art for improving flow characteristics of blood into and from a percutaneously-placed cannula.

Moreover, such cannulas are often placed with the assistance of a guidewire. Typical guidewires with curved distal tips are primarily designed for use in transcatheter aortic valve replacements. For example, the Safari, double-curved lunderquist, confida brecker curve, and Amplatz curved guidewires are all forms of known guidewires. Though the distal curves have increased flexibility in relation to the rest of the guidewire, they retain the shape of the curve against the compression of a contracting left ventricle. As such, they remain relatively stiff and have a curved radius (e.g., 3 cm; extra-small Safari and confida brecker) that is too large for placement in the majority of vessels (e.g., right or left pulmonary artery have average diameters <2.5 cm). Thus, the relative stiffness of such wires and undesirable shape retention can cause the vessel to undesirably stretch and can cause a pulmonary artery catheter to coil.

For example, some typical wires are made to retain shape against a contracting ventricle, and as such have relatively stiff curves. One of the issues such typical wires is it causes the pulmonary artery catheter to coil as the wire is passed through. Also, the smallest curve on such wires is larger than the average right or left pulmonary artery. Using a wire with such a curve that is that large and stiff in the thin-walled pulmonary arteries could potentially stretch the vessel proving dangerous, such that there remains a need in the art for improved guidewires used for placement of cannulas and catheters into a patient’s pulmonary artery.

### SUMMARY OF THE INVENTION

Disclosed herein is an improved modular cannula for drainage and return of fluids from and to a body, such as the body of a patient, that is adjustable to enable an operator to achieve desired flow characteristics during such fluid drainage and return procedures. In accordance with certain aspects of an embodiment of the invention, a modular, dual-lumen cannula system is provided that has an outer drainage cannula and an inner return cannula that is moveable with respect to the outer drainage cannula. By enabling movement of the inner return cannula with respect to the outer drainage cannula, a distance between return outlet ports at the distal end of the return cannula and drainage ports at the distal end of the drainage cannula may be manually adjusted. Such adjustable distance allows for optimal positioning of both the drainage and return ports that are particularly adapted to an individual patient’s physiology, in turn enabling optimal fluid flow characteristics to be achieved regardless of that patient physiology.

In accordance with certain aspects of an embodiment of the invention, a modular dual-lumen cannula system is provided, comprising: a first drainage cannula having a drainage cannula distal end and a drainage cannula proximal end, and a drainage cannula tip at the drainage cannula distal end, the drainage cannula tip having a drainage tip opening at the drainage cannula distal end, wherein the drainage cannula tip tapers from a first drainage cannula diameter to a second drainage cannula diameter; a plurality of drainage ports through a circumferential wall of the first drainage cannula proximal to the drainage cannula tip; the drainage cannula proximal end having a first drainage branch configured for connection to fluid drainage tubing and a second return cannula branch; a hemostatic access port affixed to a proximal end of the second return cannula branch; a second return cannula having a return cannula distal end and a return cannula proximal end configured for connection to fluid return tubing, and a return cannula tip at the return cannula distal end, the return cannula tip having a tapering portion tapering from a first return cannula diameter to a second return cannula diameter; and a plurality of return ports through a circumferential wall of the return cannula tip; wherein the second return cannula extends through the hemostatic access port into and coaxially through a drainage lumen defined by an interior of the drainage cannula and extending out from the drainage lumen through the drainage cannula tip; wherein the second return cannula is moveable through the hemostatic access port and the drainage lumen such that a distance between the drainage ports and the return ports may be manually adjusted; and wherein the hemostatic access port is adjustable to selectively enable and prevent movement of the second return cannula with respect to the first drainage cannula.

Still other aspects, features and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized. The present invention is illustrated by way of example, and not by way of limitation, in the figures of the accompanying drawings, in which like reference numerals refer to similar elements, and in which:

FIG. 1 is a front view of modular dual-lumen cannula in accordance with certain aspects of an embodiment of the invention.

FIG. 2 is a front view of an outer drainage cannula for use with the modular dual-lumen cannula of FIG. 1.

FIG. 3 is a front view of an inner return cannula for use with the modular dual-lumen cannula of FIG. 1.

FIG. 4 is a side view of an outlet tip of the inner return cannula of FIG. 3.

FIG. 5 is a front view of an inner return cannula for use with the modular dual-lumen cannula of FIG. 1.

FIG. 6 is a side view of an outlet tip of the inner return cannula of FIG. 3 according to further aspects of the invention.

FIG. 7 is a side view of a protective cover for use with the modular dual-lumen cannula of FIG. 1.

FIG. 8 is a perspective view of the protective cover of FIG. 7.

FIG. 9 a side view of hinged clasps for use with the modular dual-lumen cannula of FIG. 1 in which the hinged clasps are in an open position.

FIG. 10 a side view of hinged clasps for use with the modular dual-lumen cannula of FIG. 1 in which the hinged clasps are in a closed position.

FIG. 11 is a schematic view of the modular dual-lumen cannula of FIG. 1 being placed inside of a patient.

FIG. 12 is a schematic view of the modular dual-lumen cannula of FIG. 1 placed inside of a patient at a desired location to enable drainage through the outer drainage cannula and return through the inner return cannula.

FIG. 13 is a schematic view of a curved guidewire suitable for use with the modular dual-lumen cannula of FIG. 1.

### DETAILED DESCRIPTION OF THE PREFERRED EMBODIMENTS

The following detailed description is provided to gain a comprehensive understanding of the methods, apparatuses and/or systems described herein. Various changes, modifications, and equivalents of the systems, apparatuses and/or methods described herein will suggest themselves to those of ordinary skill in the art.

Descriptions of well-known functions and structures are omitted to enhance clarity and conciseness. The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. Furthermore, the use of the terms a, an, etc. does not denote a limitation of quantity, but rather denotes the presence of at least one of the referenced items.

The use of the terms “first”, “second”, and the like does not imply any particular order, but they are included to identify individual elements. Moreover, the use of the terms first, second, etc. does not denote any order of importance, but rather the terms first, second, etc. are used to distinguish one element from another. It will be further understood that the terms “comprises” and/or “comprising”, or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Although some features may be described with respect to individual exemplary embodiments, aspects need not be limited thereto such that features from one or more exemplary embodiments may be combinable with other features from one or more exemplary embodiments.

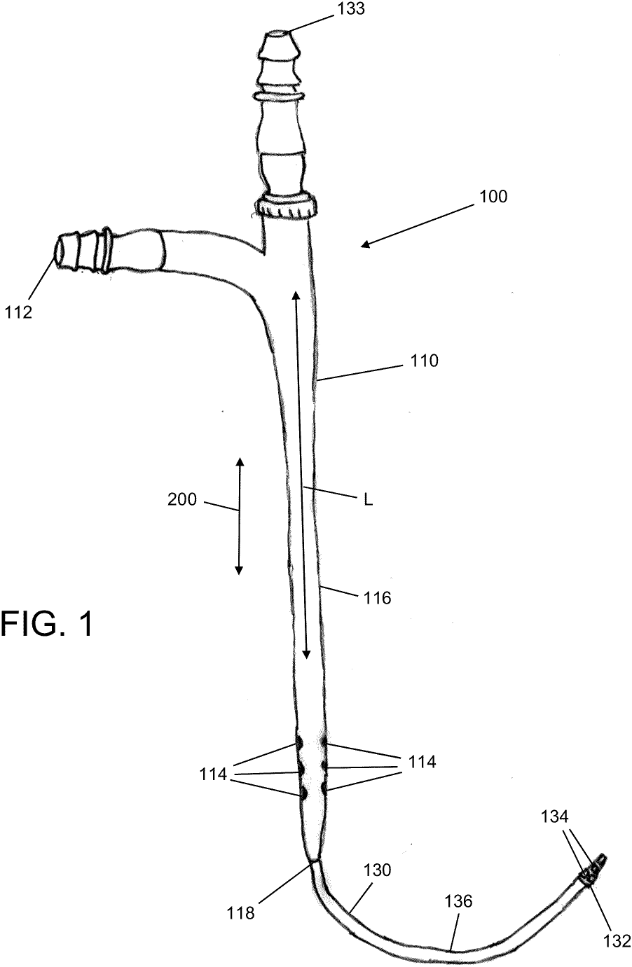

As shown in FIG. 1, and in accordance with certain aspects of an embodiment of the invention, a modular dual-lumen cannula 100 is provided that is particularly configured to improve flow and drainage of a percutaneously-placed drainage and return cannula. A dual-lumen cannula configured in accordance with at least certain aspects of the invention may be useful in a variety of procedures and devices, such as (by way of non-limiting example) with ventricular assist devices in patients with right ventricular failure who require right ventricular support, or with pump-oxygenators for ECMO in patients with pulmonary failure in need of respiratory support. For example, a dual-lumen cannula configured in accordance with aspects of the invention can be used in centers that perform cardiac surgery and provide ECMO support.

With continuing reference to FIG. 1, modular dual-lumen cannula 100 includes an outer drainage cannula 110 and an inner return cannula 130, such as an arterial return cannula, that coaxially extends through the length of and extends from the distal end of drainage cannula 110 along a longitudinal axis L of outer drainage cannula 110. Inner return cannula 130 enters into outer drainage cannula 110 through a hemostatic access port 150 that is affixed to one branch of the proximal end of outer drainage cannula 110. As discussed in greater detail below, access port 150 is configured to sealingly hold inner return cannula 130 in a fixed position (when tightened) with respect to outer drainage cannula 110 to prevent leakage of fluid from the interior of drainage cannula 110 around the point of entry of inner return cannula 130, and in certain preferred embodiments is configured to prevent movement of inner return cannula 130 with respect to outer drainage cannula 110. In accordance with a particular aspect of the invention and as discussed in greater detail below, when not locked in position by access port 150, inner return cannula 130 is variably positionable with respect to drainage cannula 110, and more particularly inner return cannula 130 may move through drainage cannula 110 along longitudinal axis L of drainage cannula 130 (in the direction of arrow 200 in FIG. 1) in order to enable an operator to vary the distance between drainage ports 114 on outer drainage cannula 110 and outlet ports 134 on inner return cannula 130 in order to generate optimal fluid flow conditions that are particularly adapted for a given patient’s anatomy. With such configuration, outer drainage cannula 110 may be held stationary while inner return cannula 130 is repositioned in the patient’s body, or inner return cannula 130 may be held stationary while outer drainage cannula 110 is repositioned in the patient’s body.

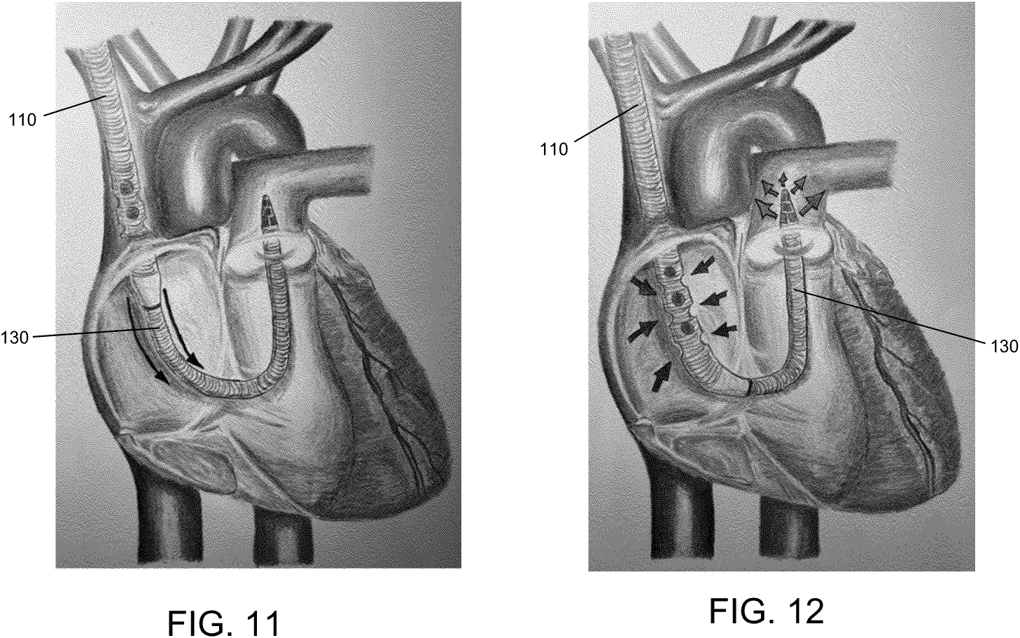

As best viewed in FIGS. 11 and 12, a modular, dual-lumen cannula 100 configured in accordance with aspects of the invention may be placed percutaneously to position a tip 118 of drainage cannula 110 into a patient’s right atrium for draining blood, with the tip 132 of inner return cannula 130 extending into the patient’s pulmonary artery. Such a configuration may be used with ventricular assist devices for right ventricular support or pump-oxygenators for extracorporeal membrane oxygenation (ECMO) and respiratory support.

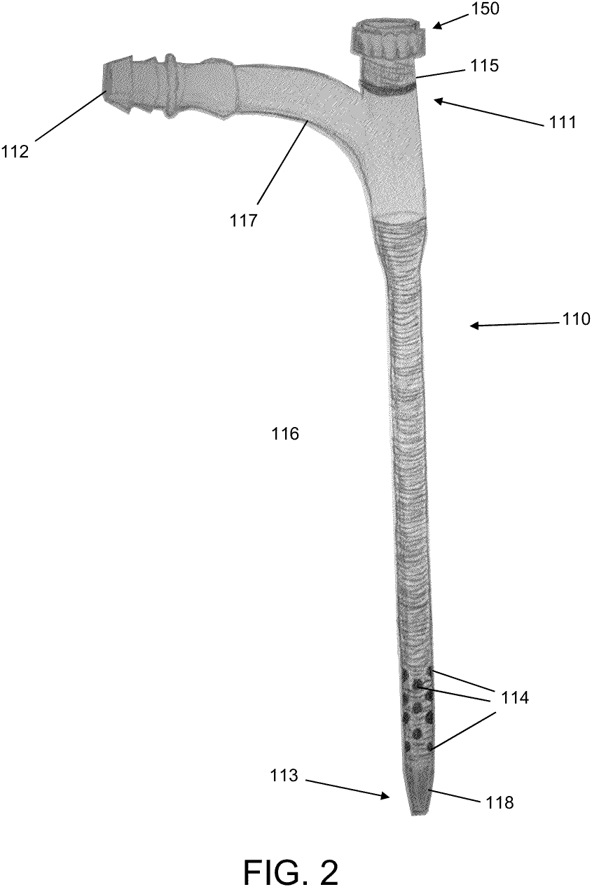

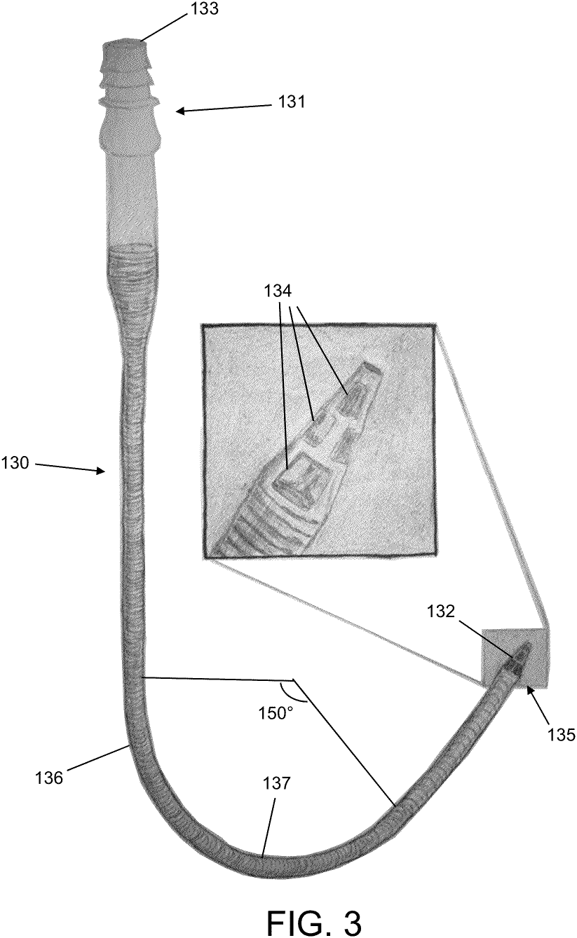

As shown in FIG. 1 and FIG. 2, outer drainage cannula 110 has a body 116 manufactured from a suitable medical-grade material (such as by way of non-limiting example polyurethane or similarly configured materials as will readily occur to those skilled in the art) that defines an internal drainage lumen that extends from the distal tip 118 of outer drainage cannula 110 to drainage cannula outlet 112. Likewise and as shown in FIG. 1 and FIG. 3, inner return cannula 130 has a body 136 again manufactured from a suitable medical-grade material (such as by way of non-limiting example polyurethane or similarly configured materials as will readily occur to those skilled in the art) that defines an internal return or infusion lumen that extends from return cannula inlet 133 to return cannula outlet tip 132. The outer diameter of body 136 of inner return cannula 130 is less than the inner diameter of body 116 of outer drainage cannula 110, such that when modular dual-lumen cannula 100 is assembled, the drainage lumen in drainage cannula 110 comprises the annular space between the exterior of body 136 of inner return cannula 130 and the interior of body 116 of outer drainage cannula 110.

Referring again to FIGS. 1 and 2, drainage cannula 110 has a drainage cannula proximal end 111 and a drainage cannula distal end 113. As used herein, the term “proximal end” is intended to refer to that portion of an element that is opposite the end that is inserted into a patient, and the term “distal end” is intended to refer to that portion of an element that coincides with the end that is inserted into the patient. Proximal end 111 of drainage cannula 110 defines separate branches 115 and 117 that generally form a Y-like connector portion. Branch 115 of proximal end 111 includes hemostatic access port 150 that allows the insertion of inner return cannula 130 that is preferably configured to extend to a patient’s pulmonary artery, or an introducer for placement of drainage cannula 110 into the patient’s vasculature. Likewise, branch 117 of proximal end 111 leads to drainage cannula outlet 112, which is configured to receive tubing allowing for drainage of blood from the patient through drainage cannula 110 for circulatory support and ECMO applications.

Distal end 113 of drainage cannula 110 holds distal tip 118 that defines a tapering portion of body 116 of drainage cannula 110. The tapering portion of body 116 defined by distal tip 118 tapers from the full diameter of body 116 of drainage cannula 110 to a smaller diameter that approximates the outer diameter of inner return cannula 130. In a particularly preferred configuration, distal tip 118 of drainage cannula 110 may taper along a length of drainage cannula 110, such as over a length of 2-3 cm or over a length of 3-5 cm. Further in a particularly preferred configuration, distal tip 118 of drainage cannula 110 may taper, for example, from a 31 French diameter to an 18 French diameter, thus allowing placement of an 18 French diameter inner return cannula 130. Likewise in certain configurations, distal tip 118 may taper from a 19 French diameter to a 12 French diameter. Distal tip 118 of drainage cannula 110 is thus configured to coaxially center inner return cannula 130 at distal tip 118. In combination with access port 150 (through which inner return canula 130 enters into drainage cannula 110), inner return cannula 130 is thus held coaxially within drainage cannula body 116 through the length of drainage cannula body 116. Moreover, in this configuration, both distal tip 118 of drainage cannula 118 and hemostatic access port 150 serve as fixation points that substantially maintain a central coaxial alignment between outer drainage cannula 110 and inner return cannula 130, thus preventing “free floating” of inner return cannula 130 either within drainage cannula body 116 or at distal tip 118 of drainage cannula 110. In this manner, modular dual-lumen cannula 100 is particularly configured to maintain a central coaxial alignment between outer drainage cannula 110 and inner return cannula 130 to, in turn, decrease the risk of turbulent flow and subsequent risk of hemolysis and thrombosis, as compared to previously known cannulas that are positioned against or adjacent to an inner wall of an outer cannula.

Just proximal to distal tip 118 of drainage cannula 110 (and thus still generally located at distal end 113 of drainage cannula 110) are drainage ports 114. Drainage cannula 110 is provided a length suitable to position drainage ports 114 in a patient’s right atrium when the patient’s circulatory system is accessed, for example, through percutaneous insertion into the patient’s jugular vein. Drainage ports 114 preferably are arranged around the circumference of body 116 of drainage cannula 110, and may be elliptical in shape, including circular. However, drainage ports 114 may likewise be provided in alternative shapes, such as (by way of non-limiting example) generally trapezoidal or rectangular.

In a particularly preferred configuration, drainage cannula 110 may have an insertable cannula length (i.e., a length of that portion of drainage cannula 110 having an outer diameter suitable for placement into the patient’s circulatory system and thus distal to the Y-shaped connectors and wider diameter portions of proximal end 111 of drainage cannula 110) that minimizes the portion of drainage cannula that is external to the percutaneous placement. For example, the insertable length of drainage cannula 110 may be approximately 25 cm, such that distal tip 118 of drainage cannula 110 is positioned within the right atrium of most patients when percutaneously inserted into the patient’s jugular vein (e.g., about 5 cm distal to a pulmonary valve), and minimizing an external portion of the drainage cannula 110 that extends outward from the percutaneous point of entry. Minimizing the external portion of drainage cannula 110 in this manner reduces resistance across the length of drainage cannula 110 and generally improves safety when using modular, dual-lumen cannula 100.

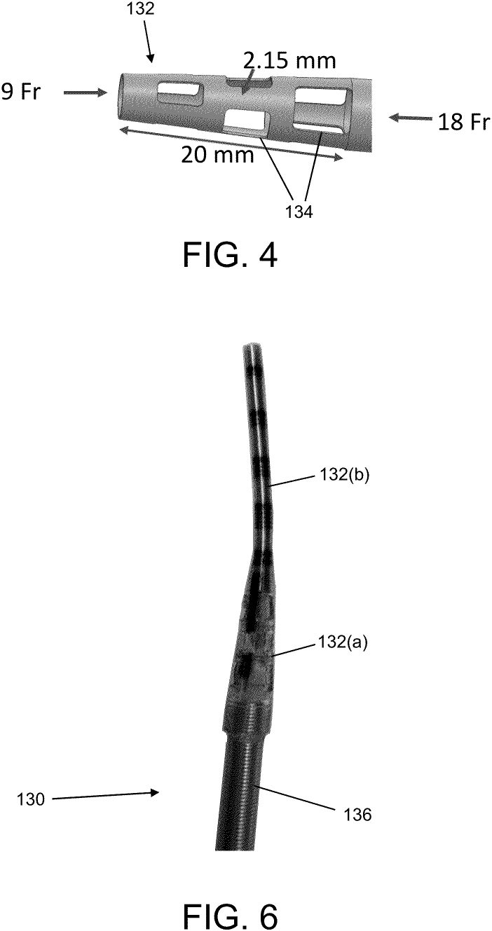

Next and with continuing reference to FIGS. 1 and 3, inner return cannula 130 has a proximal end 131 and a distal end 135. Inner return cannula 130 has a length greater than the length of outer drainage cannula, and more particularly preferably has an insertable length of approximately 50 cm. Proximal end 131 of return cannula 133 is configured to receive tubing allowing for the supply of blood to return cannula 130 and ultimately to the patient. As will be discussed in greater detail below, return cannula 130 is likewise configured to engage with hemostatic access port 150 on drainage cannula 110 to provide an entry point that is sealed against fluid flow around return cannula 130, but that allows for varied longitudinal positioning of return cannula 130 (i.e., movement of return cannula 130 along axis L of drainage cannula 110). Distal end 135 of return cannula includes outlet tip 132. Outlet tip 132 tapers from a diameter equal to the diameter of cannula body 136 down to a smaller diameter at the distal end of outlet tip 132. In a particularly preferred configuration, outlet tip 132 may taper along a length from return cannula body 136 to the distal end of outlet tip 132 of 20 mm, and from a widest diameter of 18 French to a reduced diameter of 9-10 French at the distal end of outlet tip 132. Outlet tip 132 also includes outlet ports 134 extending around the circumference of outlet tip 132 in multiple rows between return cannula body 136 and the distal end of outlet tip 132. Again in a particularly preferred configuration, each of outlet ports 134 preferably has a length dimension of 2.15 mm and a width dimension that is less than the length dimension, with the width dimension preferably being reduced for successive rows of outlet ports as they approach the distal end of outlet tip 132. FIG. 4 provides a close-up view of outlet tip 132 with such exemplary dimensions.

In certain configurations, modular dual-lumen cannula 100 may be configured with a preformed bend to facilitate placement at a desired location in the patient’s body. For example and with continuing reference to FIG. 3, return cannula 130 may be formed with a preformed bend 137 that facilitates placement and maintenance of the outlet tip 132 of the return cannula 130 in the patient’s pulmonary artery. Thus and in certain configurations, return cannula 130 may have a preformed bend 137 between a percutaneous entry (e.g., at a site accessing the patient’s jugular vein) and a distal point of the return cannula 130. In an exemplary configuration, return cannula 130 may employ such a preformed bend 137 along a length of, for example, 25 cm of the return cannula 130 closer to the distal end 135 of return cannula 130 than the proximal end 131 of return cannula 130. Further, such preformed bend may follow a curvilinear path, such as exhibiting an angle of 120 - 160 degrees between the percutaneous entry and the distal end 135, and more preferably an angle of 150 degrees. In this configuration, the outlet tip 132 of inner return cannula 130 may be placed in the patient’s pulmonary artery with a desired position of the distal tip being determined by the operator (e.g., either in the main pulmonary arterial trunk, in the right main pulmonary artery, or in the left main pulmonary artery). Typical bends that are substantially less than 120 degrees may exert more outward force onto intracardiac structures, while other typical bends substantially greater than 160 degrees may result in less desirable flow, such as preferential flow towards the right main pulmonary artery.

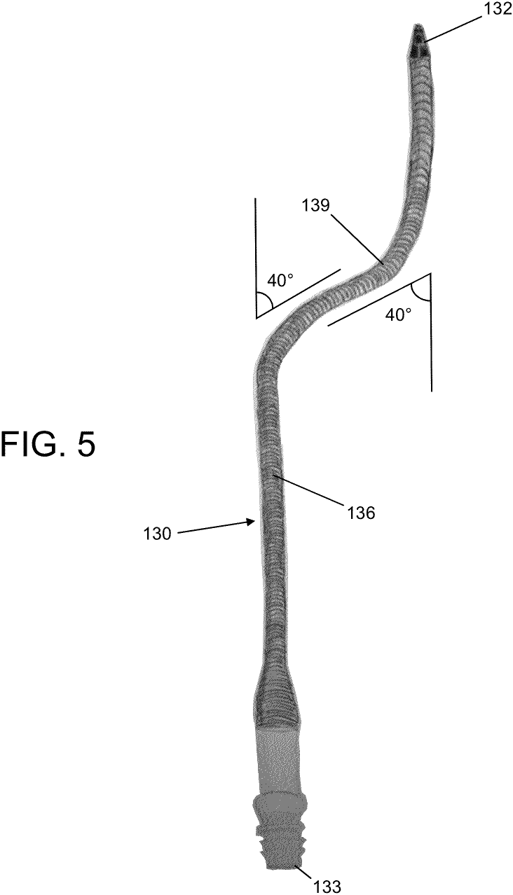

In certain configurations, the modular dual-lumen cannula may enter percutaneously from a common femoral vein. In such applications and as shown in FIG. 5, a distal 25 cm portion of return cannula 130 may be preformed to have an S-like shaped curve 139 exhibiting two 40 degree turns. In this configuration with a femoral vein percutaneous entry, return cannula 130 may be placed into a desired position without exerting undesired pressure on intracardiac structures, e.g., the patient’s tricuspid valve, right ventricle, pulmonary valve, pulmonary arterial valve, or the like.

In certain configurations, outlet tip 132 may be configured as shown in FIG. 6. In this configuration, an outlet tip 132(a) is located again at the distal end of return cannula 130, but outlet tip 132(a) is provided a distal extension 132(b) exhibiting a smallest diameter of outlet tip 132(a) and being configured to sit within either the right or left pulmonary artery, which may aid in properly locating the position of outlet tip 132(a) on radiograph. In an exemplary configuration, distal extension 132(b) may have a length of 4.5 cm and a diameter of 10 French. Such an outlet tip 132(a) including distal extension 132(b) may be particularly beneficial in optimizing fluid flow conditions, and particularly in providing less turbulent flow into the patient’s pulmonary artery than previously available constructions. Outlet tip 132(a) may, for instance, be placed in the main trunk of the patient’s pulmonary artery with the outlet from extension 132(b) at the left and right branches of the patient’s pulmonary artery, which distributed outlets have shown to improve upon flow conditions (i.e., provide less turbulent flow conditions) in comparison to previously known configurations.

In certain configurations, one or both of outer drainage cannula 110 and inner return cannula 130 may be configured having varying flexibility along their lengths, particularly in comparison to typical wire-reinforced percutaneously-placed cannulas. By way of non-limiting example, either or both of outer drainage cannula 110 and inner return cannula 130 may have variable or non-uniform reinforcement to vary rigidity using various wire reinforcement, materials of varying stiffness, and/or sections of varying thickness, all of which may be selected and configured to assist in placement of the components of modular, dual-lumen cannula 100 in a desired position within the patient’s body. Thus, dual-lumen cannula 100 may be placed into such a desired position without exerting undesired pressure on intracardiac structures, e.g., the patient’s tricuspid valve, right ventricle, pulmonary valve, pulmonary arterial valve, or the like.

As mentioned above, hemostatic access port 150 is configured to sealingly hold inner return cannula 130 in a fixed position (when tightened) with respect to outer drainage cannula 110 to prevent leakage of fluid from the interior of drainage cannula 110 around the point of entry of inner return lumen 130, and in certain preferred embodiments is configured to prevent movement of inner return cannula 130 with respect to outer drainage cannula 110. Modular dual-lumen cannula 100 is thus configured to reduce the likelihood of formation of a pulmonary embolism. For example, access port 150 may comprise a valved access port, such as a valved port incorporating a Touhey-Borst type valve construction (the construction of which is well known to those skilled in the art) that will enable insertion of return cannula 130 through the valved access port 150 and into outer drainage cannula 110, while enabling sealing of the valved access port 150 against the outer body of inner return cannula 130. Thus, when the distal end 135 of inner return cannula 130 has reached the intended position inside of a particular patient’s pulmonary artery, the valved access port 150 may be tightened to a desirable tension to maintain hemostasis and to prevent air entrainment, such as during use with an ECMO / CPB system. In certain configurations, such a valved access port may likewise comprise a quick-connect valve assembly. Valved access port 150 may further be configured to reduce the likelihood of kinking of the inner return cannula 130 by preventing overtightening, such as through use of a torque-ratcheted mechanism configured to prevent overtightening (the construction of which will be apparent to those skilled in the art). Further, in certain configurations modular dual-lumen cannula 100 may be configured to prevent an area of stagnation between the insertion point of inner return cannula 130 at access port 150 and the main body of outer drainage cannula 110, such as by providing an internal diaphragm (not shown and of standard configuration) in line with the lumen of outer drainage cannula 110 through which internal return cannula passes. In such construction, the diaphragm is preferably positioned as close as possible to the bifurcation of the drainage lumen and the access port lumen inside of outer drainage cannula 110.

Still further, exemplary embodiments may include indicators providing information on different properties of the modular dual-lumen cannula 100. By way of non-limiting example, visual markers (not shown) may be provided indicating a position (e.g., open, closed, partially-open) or degree of rotation of the valve, or an extent to which the valve should be further tightened or loosened to achieve a particularly desired position or condition. Further, visual markers may be provided that indicate a torque of a component (such as access port 150) on the cannula 100, such as the current torque of a valved access port 150 or remaining torque that is to be applied or removed from the valved access port 150 to again achieve a particularly desired position or condition. Still further, visual markers may be provided in the form of markings on the valved access port, such as by way of non-limiting example color markings, that indicate open, closed, or partially-open states of the valved access port 150.

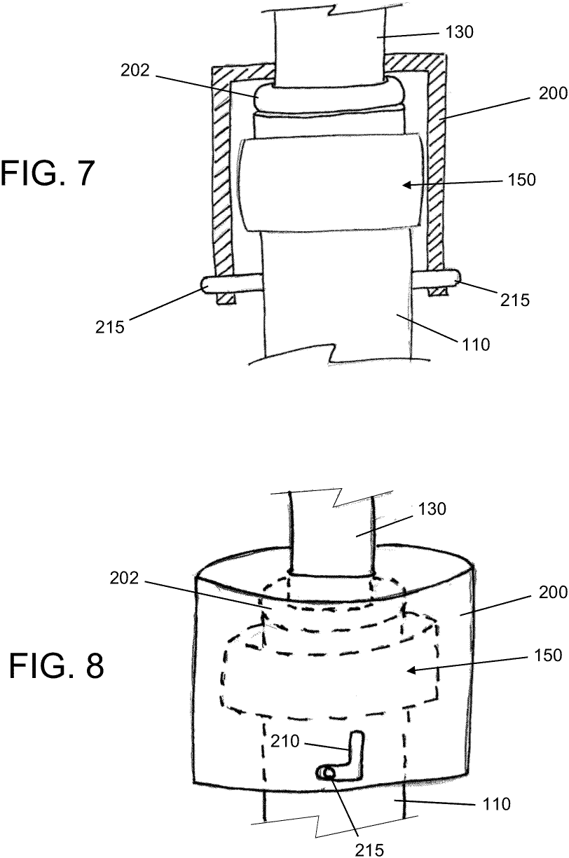

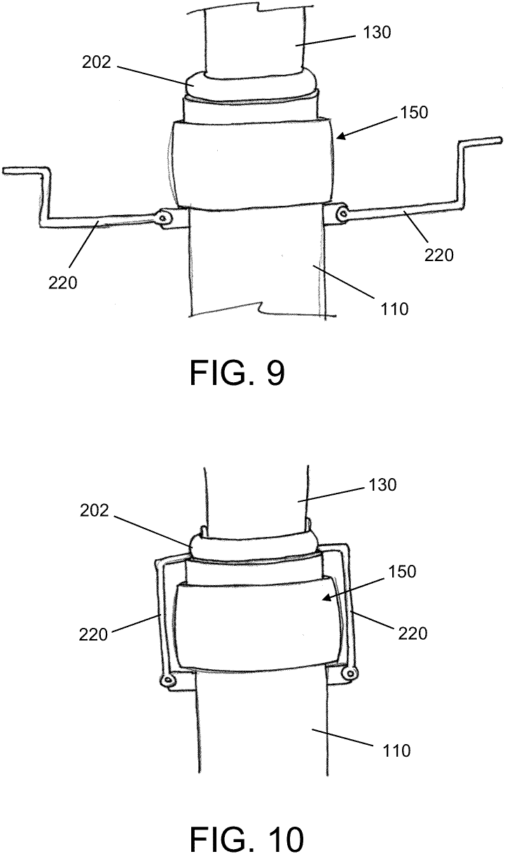

In certain configurations, additional mechanisms may be provided to reduce the likelihood of inadvertent adjustment of valved access port 150 (e.g., loosening of the valved access port after both the outer drainage cannula 110 and the inner return cannula 130 are in their intended positions). In certain cases, undesirable loosening could lead to blood loss or air entrainment into an ECMO / CPB system. For example, and with particular reference to FIGS. 7 and 8, a cap 200 may be provided having an opening in the top wall of the cap through which inner return cannula 130 may pass for entry into valved access port 150 of outer drainage cannula 110. Additionally, a compressible sealing member 202, such as a gasket, O-ring, or similarly configured member, may extend around inner return cannula 130 at the point at which inner return cannula 130 enters into valved access port 150. When cap 200 is pushed downward (i.e., toward valved access port 150), compressible sealing member 202 is compressed between cap 200 and the top of valved access port 150, adding additional sealing between inner return cannula 130 and outer drainage cannula 110. In an exemplary configuration, cap 200 may include a slot 210 that may engage a pin 215 or similar member extending outward from a sidewall of outer drainage cannula 110, such that when cap 200 is pushed downward and turned about the longitudinal axis L of outer drainage cannula 110, a lower portion of groove 210 receives pin 215 to lock cap 200 in place in a compressive state against compressible sealing member 202. Optionally, a still further outer cap (not shown) may be provided over cap 200 (again through which inner return cannula 130 may pass) to cover the full protective cap 200 and likewise prevent inadvertent dislodging of cap 200 from outer drainage cannula 110. As a further example and with reference to FIGS. 9 and 10, hinged clips 220 may be pivotably mounted on the outer wall of outer drainage cannula 110, and may be pivoted upward (as particularly shown in FIG. 10) and held in place by compressible sealing member 202 (with the ends of each clip held between the interior of compressible sealing member 202 and the exterior of inner return cannula 130) to further hold inner return cannula 130 in the intended position with respect to outer drainage cannula 110. Again, a still further outer cap (not shown) may be provided over hinged clips 220 once in position against inner return cannula 130 to cover the full entry point of inner return cannula 130 into outer drainage cannula 110. As still yet a further example, a knob of valved access port 150 may be configured with a faceted body (e.g., a hexagonal circumference), and a mating outer cap having a complementary faceted interior may again be provided over such faceted knob and locked in place using the foregoing assemblies to directly prevent rotation of the knob of valved access port 150.

With regard to still further aspects of an embodiment, outer drainage cannula 110 may include features that are particularly configured to reduce the likelihood of dislodgment and undesirable movement. For example, outer drainage cannula 110 may extend proximally from the connector (or Y-like split, discussed above) in a substantially transverse direction (i.e. along a transverse plane). As a further example, the connector may be angled to extend the drainage cannula 110 away from a patient’s head or neck. With this configuration, the drainage cannula 110 does not attach to the patient’s head or neck and is therefore less likely to cause discomfort or be dislodged by the patient.

With regard to still further aspects of an embodiment, modular, dual-lumen cannula 100 may be configured to reduce the likelihood of fluid loss within the cannula. For example, modular, dual-lumen cannula 110 may be configured to receive a clamp to control fluid flow. A discrete proximal portion of outer drainage cannula 110 may be configured without wire reinforcement to aid in clamping the outer drainage cannula 110, such as with typical medical utility clamps. Thus, a proximal portion of outer drainage cannula 110 may be clamped when the valve is open to prevent blood loss or air entrainment.

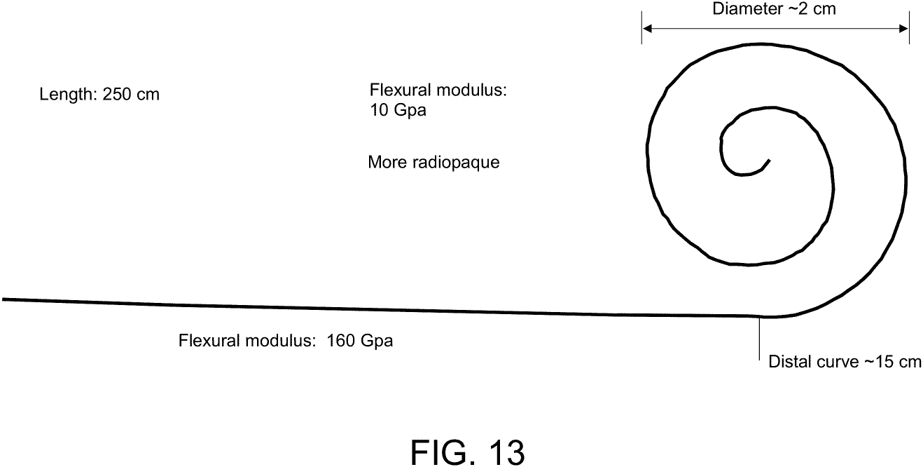

Next and in accordance with still further aspects of the invention, and with particular reference to FIG. 13, a soft-tip curved guidewire is provided that is configured to be percutaneously placed into intravascular structures, such as modular, percutaneous dual-lumen cannula 100. The guidewire can serve as a guide rail for percutaneous insertion of intravascular devices, including cannulas for extracorporeal membrane oxygenation (ECMO), where there is concern for potential injury of the distal intravascular structure. Thus, a wire according to certain aspects of the invention that is configured to reduce the likelihood of injury to the pulmonary artery can also provide a rail stiff enough to advance the cannula because of a curved distal end for reduced intravascular trauma.

In an exemplary configuration, the distal 15 cm of the guidewire are curved into a spiral-like shape with a diameter of approximately 2 cm. This configuration reduces the risk that the guidewire will inadvertently advance into branch vessels once in the desired location, compared to typical guidewires. For example, if the desired location for the end of the guidewire is in the right pulmonary artery, the curved distal end will prevent the guidewire from advancing into the branch pulmonary arteries. This reduces the risk of intravascular trauma to branch vessels and better stabilizes the distal portion of the guidewire.

With regard to further aspects of an embodiment, the guidewire may be configured for increased navigability during a procedure. For example, the distal 15 cm of the guidewire has a stiffness that is greatly reduced compared to the rest of the guidewire. This reduced stiffness or rigidity allows the distal end of the guidewire to navigate turns through guide catheters that take tortuous intravascular paths without requiring the use of exchange catheters. For example, a balloon-tipped pulmonary artery catheter that is positioned in the pulmonary artery from an upper body approach takes a turn >180° as it traverses the tricuspid and pulmonic valve. A stiff wire placed directly through this catheter would commonly displace the catheters towards the right ventricular apex as it approaches that turn, potentially causing the tip of the pulmonary artery catheter to prolapse back out of the pulmonary artery. This situation may require a more flexible wire with a stiffer exchange catheter. The increased flexibility of the distal 15 cm of the soft-rip curved guidewire allows the distal end to navigate the turn and reduces the likelihood of displacing the pulmonary arterial catheter. The stiffer portion of the wire provides a guide rail for an intravascular device to be placed (e.g. ECMO cannula) without requiring an exchange of the guidewire.

The soft-tip curved guidewire is intended for use in situations where there is concern for potential injury of distal intravascular structures, and there is difficulty in navigating to the desired area. One immediate application is in the percutaneous placement of devices that require a guide rail to the pulmonary artery. Examples include percutaneous placement of a cannula into the pulmonary artery for patients who are in need of right ventricular support or ECMO, or percutaneous pulmonary valve replacement in patients with pulmonary valvular disease.

In an exemplary configuration, the guidewire is approximately 250 cm in length with a flexural modulus of approximately 160 gigapascals. The guidewire can have a stiffness similar to a Lunderquist wire. The guidewire can also have a curved tip that measures 15 cm in length, a diameter of 2 cm, a stiffness of 10 gigapascal (such as a typical j-wire), and is more radiopaque than the rest of the wire.

Thus, a guidewire configured in accordance with at least certain aspects of the invention allows the guidewire to pass through the pulmonary artery catheter more easily compared to typical guidewires. A floppier (distal) end of the guidewire will reach the pulmonary valve before the stiffer (more rigid) proximal portion has to take the turn in the right RA-RV, reducing the likelihood of displacing the pulmonary artery catheter towards the RV apex and prolapsing it out of the pulmonary artery, and increasing the trackability of the wire.

The curve is configured to reduce the risk of perforation compared to a straight tip wire and prevents the wires from going down into the branch vessels compared to typical wires. The guidewire wire also has sufficient stiffness to provide a rail for advancing a cannula. The guidewire also allows pulmonary artery cannula placement, and may additionally be useful for transcatheter pulmonary artery interventions, such as valve replacements.

Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept. Thus, it should be understood, therefore, that the invention may be practiced otherwise than as specifically set forth herein.

What is claimed is:

1. A modular dual-lumen cannula system, comprising:
a first drainage cannula having a drainage cannula distal end and a drainage cannula proximal end, and a drainage cannula tip at the drainage cannula distal end, the drainage cannula tip having a drainage tip opening at the drainage cannula distal end, wherein the drainage cannula tip tapers from a first drainage cannula diameter to a second drainage cannula diameter;
a plurality of drainage ports through a circumferential wall of the first drainage cannula proximal to the drainage cannula tip;
the drainage cannula proximal end having a first drainage branch configured for connection to fluid drainage tubing and a second return cannula branch;
a hemostatic access port affixed to a proximal end of the second return cannula branch;
a second return cannula having a return cannula distal end and a return cannula proximal end configured for connection to fluid return tubing, and a return cannula tip at the return cannula distal end, the return cannula tip having a tapering portion tapering from a first return cannula diameter to a second return cannula diameter; and
a plurality of return ports through a circumferential wall of the return cannula tip;
wherein the second return cannula extends through the hemostatic access port into and coaxially through a drainage lumen defined by an interior of the drainage cannula and extending out from the drainage lumen through the drainage cannula tip;
wherein the second return cannula is moveable through the hemostatic access port and the drainage lumen such that a distance between the drainage ports and the return ports may be manuualy adjusted; and
wherein the hemostatic access port is adjustable to selectively enable and prevent movement of the second return cannula with respect to the first drainage cannula.

2. The modular dual-lumen cannula system of claim 1, wherein the first diameter of the second rturn cannula approximates the second diameter of the first drainage cannula.

3. The modular dual-lumen cannula system of claim 2, wherein the first diameter of the first drainage cannula is 19-31 French.

4. The modular dual-lumen cannula system of claim 3, wherein the second diameter of the first drainage cannula is 12-18 French.

5. The modular dual-lumen cannula system of claim 1, wherein the hemostatic access port and the drainage tip opening at the drainage tip distal end are configured to coaxially hold the return cannula within the drainage lumen of the drainage cannula.

6. The modular dual-lumen cannula system of claim 1, wherein the drainage cannula has an insertable length of 25 cm.

7. The modular dual-lumen cannula system of claim 6, wherein the return cannula has an insertable length of 50 cm.

8. The modular dual-lumen cannula system of claim 1, wherein the return cannula tip tapers along a length of 20 mm of the return cannula tip.

9. The modular dual-lumen cannula system of claim 8, wherein the return cannula tip tapers from a diameter of 18 French to a diameter of 9-10 French.

10. The modular dual-lumen cannula system of claim 1, wherein the return ports are arranged in multiple rows around a circumference of the return cannula tip.

11. The modular dual-lumen cannula system of claim 10, wherein each return port has a longest length dimension of 2.15 mm and a width dimension that is less than the length dimension.

12. The modular dual-lumen cannula system of claim 11, wherein the width dimension declines for successive rows of return ports as they approach the distal end of the return cannula tip.

13. The modular dual-lumen cannula system of claim 1, wherein the return cannula is formed with a preformed bend between a return cannula proximal end and the return cannula distal end.

14. The modular dual-lumen cannula system of claim 13, wherein said preformed bend is located along a 25 cm length of said return cannula.

15. The modular dual-lumen cannula system of claim 14, wherein said 25 cm length is closer to the return cannula distal end than the return cannula proximal end.

16. The modular dual-lumen cannula system of claim 14, wherein the preformed bend follows a curvilinear path and exhibits an angle of 120 - 160 degrees.

17. The modular dual-lumen cannula system of claim 14, wherein the preformed bend follows an S-shaped curve and exhibits two 40 degree bends.

18. The modular dual-lumen cannula system of claim 1, the return cannula further comprising a distal extension extending from a distal end of the return cannula tapering portion and having an open distal extension outlet at an end of the distal extension.

19. The modular dual-lumen cannula system of claim 18, wherein the distal extension has a length of 4.5 cm.

20. The modular dual-lumen cannula system of claim 18, wherein the distal extension has a diameter matching the second diameter of the return cannula tapering portion.

21. The modular dual-lumen cannula system of claim 1, wherein the hemostatic access port further comprises a valved access port.

22. The modular dual-lumen cannula system of claim 21, wherein the valved access port further comprises a Touhey-Borst valve.

23. The modular dual-lumen cannula system of claim 21, wherein the valved access port further comprises a torque-limiting valve.
